(19) United States
(12) Patent Application Publication
(10) Pub. No.: US 2021/0116453 A1
(43) Pub. Date: Apr. 22, 2021

(54) METHODS AND MATERIALS FOR ASSESSING AND TREATING LICHEN PLANUS

(71) Applicant: Mayo Foundation for Medical Education and Research, Rochester, MN (US)

(72) Inventors: Julia S. Lehman, Rochester, MN (US); Maria A. Deschaine, Rochester, MN (US)

(21) Appl. No.: 17/070,425

(22) Filed: Oct. 14, 2020

Related U.S. Application Data
(60) Provisional application No. 62/915,902, filed on Oct. 16, 2019.

(51) Int. Cl. G01N 33/574; A61K 31/573; A61K 31/145; A61K 31/52; A61K 31/436; A61K 31/5377

(52) U.S. Cl. CPC G01N 33/57484; A61K 31/573; A61K 31/5377; A61K 31/52; A61K 31/436; A61K 31/145

(57) ABSTRACT

This document provides methods and materials involved in identifying and treating mammals with lichen planus (LP) as having (a) an increased likelihood of developing malignancy, or (b) an increased likelihood of experiencing a benign course of the disease. For example, this document provides methods and materials involved in identifying and treating mammals with oral LP that are likely to proceed to a malignancy such as oral squamous cell carcinoma (SCC), or are likely to follow a benign course of the disease.

### CROSS-REFERENCE TO RELATED APPLICATIONS

This application claims priority from U.S. Provisional Application Ser. No. 62/915,902, filed Oct. 16, 2019. The disclosure of the prior application is considered part of (and is incorporated by reference in) the disclosure of this application.

### TECHNICAL FIELD

This document relates to methods and materials involved in identifying and treating mammals having lichen planus (LP) that are likely to proceed to malignancy or are likely to follow a benign course of the disorder. For example, this document provides methods and materials involved in identifying and treating mammals with oral LP that are likely to proceed to a malignancy such as oral squamous cell carcinoma (SCC), or are likely to follow a benign course of the disorder.

### BACKGROUND

LP is a chronic, inflammatory, autoimmune disorder that occurs when the immune system mounts an attack against cells of the mucous membranes or skin for unknown reasons. LP can cause swelling and irritation in the skin, hair, nails, and mucous membranes. On the skin, LP usually appears as purplish, itchy, flat bumps, while in the mouth, vagina, and other areas covered by a mucous membrane, LP can form lacy white patches, sometimes with painful sores. Symptoms sometimes can be managed through the use of topical numbing agents. In some cases, corticosteroids administered topically, orally, or by injection may be used to reduce the inflammation associated with LP. In some cases, immune response modulators (e.g., calcineurin inhibitors such as tacrolimus or pimecrolimus) may be administered locally or systemically to suppress or modify the body's immune response, thereby improving more severe lesions and reducing pain.

Patients with oral LP typically require regular monitoring, because they may be at risk of developing mouth cancer in the affected areas. For example, patients with oral LP are at increased risk of developing oral squamous cell carcinoma (SCC). To date, there is no reliable predictor to identify patients at higher risk to develop this complication, and frequently, the cancer is already invasive when it is detected. Further, the carcinogenic mechanism of oral LP remains obscure. In addition, in patients with severe and diffuse oral LP, it can be difficult to know when any regions have transformed to SCC.

### SUMMARY

This document is based, at least in part, on the identification of potential prognostic tissue-based biomarkers for development of malignancies in mammals having LP. These biomarkers can be useful to distinguish LP patients (e.g., oral LP patients) who are more likely to develop cancer from patients who are more likely to experience a benign course of the disease. Thus, this document provides methods and materials involved in identifying mammals with LP (e.g., humans with oral LP) as being likely to proceed to malignancy, as well as methods and materials for identifying mammals with oral LP as being likely to follow a benign course. For example, this document provides methods and materials for detecting the presence of an elevated level of one or more proteins (e.g., MYL6B, ENO3, MYH2, MYLPF, MYH6, TPM2, FLNC, RAB2A, HBB, STOML2, CRYAB, S100A7, CA1, TARDBP, RUVBL2, and/or SYNCRIP) in a biological sample (e.g., a cell-containing sample) from a mammal having oral LP, thereby identifying the mammal as being more likely to develop malignancy (e.g., SCC) than a mammal with oral LP that does not exhibit an elevated level of the one or more proteins. Conversely, this document provides methods and materials for determining that a biological sample from a mammal with oral LP does not have an elevated level of the one or more proteins (e.g., MYL6B, ENO3, MYH2, MYLPF, MYH6, TPM2, FLNC, RAB2A, HBB, STOML2, CRYAB, S100A7, CA1, TARDBP, RUVBL2, and/or SYNCRIP), thereby identifying the mammal as being less likely to develop malignancy (e.g., SCC) than a mammal with oral LP that does exhibit an elevated level of the one or more proteins. Identifying cells having or not having an elevated level of one or more of the markers listed herein can allow clinicians to proceed with proper clinical surveillance (e.g., frequency of clinical follow-ups, need for surveillance biopsies) and treatment options for oral LP patients.

This document also provides methods and materials involved in treating mammals identified as having oral LP that is likely to proceed to malignancy, or that is likely to follow a benign course. For example, this document provides methods and materials for administering topical and/or systemic immunosuppressive or immunomodulatory medications (e.g., corticosteroids, dapsone, azathioprine, tacrolimus, mycophenolate mofetil, or biologic agents), based at least in part on the malignancy risk. As described herein, mammals having oral LP who are likely to progress to malignancy (e.g., SCC) can be administered tailored therapies to achieve disease control while minimizing immunosuppression. Having the ability to use more aggressive systemic agents (e.g., azathioprine, tacrolimus, mycophenolate mofetil, or biologic agents) to achieve better disease control can allow clinicians and patients to proceed with treatment options that mitigate the risk for malignant transformation. In addition, mammals with increased risk for malignancy can undergo more regular surveillance (e.g., examination and/or biopsy of involved tissue on a more frequent basis) to detect changes of possible carcinoma development early in its course.

In a first aspect, this document features a method for identifying a mammal having LP as being at increased risk of developing cancer. The method can include identifying a mammal with LP as having cells with increased expression of one or more markers, where the one or more markers include one or more of myosin light chain 6B (MYL6B), beta-enolase 3 (ENO3), myosin-2 (MYH2), myosin regulatory light chain 2 (MYLPF), myosin-6 (MYH6), tropomyosin 2 (TPM2), filamin-C (FLNC), Ras-related protein Ras2A (RAS2A), hemoglobin subunit beta (HBB), stomatin-like protein 2 (STOML2), alpha-crystallin B (CRYAB), S100A7, carbonic anhydrase 1 (CA1), TAR DNA-binding protein 43 (TARDBP), ruvB-like 2 (RUVBL2), and synaptotagmin binding cytoplasmic RNA interacting protein (SYNCRIP). The mammal can be a human. The LP can be oral LP. The cells can be within a cytology brushing sample. The cells can be squamous epithelial or stromal cells affected by the LP. The identifying can include measuring the level of protein for the one or more markers, or measuring the level of mRNA for the one or more markers. The method can include identifying the mammal with oral LP as having cells with increased expression of two or more of the markers.

In another aspect, this document features a method for treating a mammal having LP. The method can include (a) identifying the mammal as having cells with increased expression of one or more markers, where the one or more markers include one or more of MYL6B, ENO3, MYH2, MYLPF, MYH6, TPM2, FLNC, RAB2A, HBB, STOML2, CRYAB, S100A7, CA1, TARDBP, RUVBL2, and SYNCRIP, and (b) administering to the mammal an immunosuppressive or immunomodulatory agent. The mammal can be a human. The LP can be oral LP. The cells can be within a cytology brushing sample. The cells can be squamous epithelial or stromal cells affected by the LP. The identifying can include measuring the level of protein for the one or more markers, or measuring the level of mRNA for the one or more markers. The method can include identifying the mammal with LP as having cells with increased expression of two or more of the polypeptides. The immunosuppressive or immunomodulatory agent can be a corticosteroid, dapsone, azathioprine, tacrolimus, mycophenolate mofetil, or a biologic agent.

This document also features a method for treating a mammal having LP, where the method includes administering, to a mammal identified as having cells with increased expression of one or more markers selected from the group consisting of MYL6B, ENO3, MYH2, MYLPF, MYH6, TPM2, FLNC, RAB2A, HBB, STOML2, CRYAB, S100A7, CA1, TARDBP, RUVBL2, and SYNCRIP, an immunosuppressive or immunomodulatory agent. The mammal can be a human. The LP can be oral LP. The cells can be squamous epithelial or stromal cells affected by the LP. The mammal with LP can have been identified as having cells with increased expression of two or more of the markers. The immunosuppressive or immunomodulatory agent can be a corticosteroid, dapsone, azathioprine, tacrolimus, mycophenolate mofetil, or a biologic agent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description, and from the claims.

### DETAILED DESCRIPTION

Patients with LP can be at risk of developing dysplasia or cancer. For example, patients with oral LP may be at higher risk of developing mouth cancer (e.g., SCC). LP patients typically require regular monitoring, as there has been no way to know which patients will go on to develop dysplasia or cancer. As described herein, however, markers for determining that a patient has an increased likelihood of malignancy have now been identified.

Thus, this document provides methods and materials for identifying and/or treating LP patients who have an increased likelihood of developing a cancer such as SCC. For example, this document provides methods and materials for identifying a mammal (e.g., a human) having oral LP as being at increased risk of progressing to malignancy. Any appropriate mammal having oral LP can be identified as being at increased risk of developing malignancy. For example, humans and other primates such as monkeys having oral LP can be identified as having an increased likelihood of progressing to malignancy. In some cases, dogs, cats, horses, cows, pigs, sheep, mice, or rats having oral LP can be identified as having an increased likelihood of progressing to malignancy, as described herein.

As described herein, a mammal (e.g., a human) with oral LP can be identified as having an increased likelihood of developing malignancy by detecting cells with increased levels of one or more markers (e.g., mRNAs or polypeptides). Examples of markers (e.g., mRNAs or polypeptides) that can be evaluated and used to classify a mammal (e.g., a human) as having or not having an increased likelihood of developing malignancy include, without limitation, myosin light chain 6B (MYL6B), beta-enolase 3 (ENO3), myosin-2 (MYH2), myosin regulatory light chain 2 (MYLPF), myosin-6 (MYH6), tropomyosin 2 (TPM2), filamin-C (FLNC), Ras-related protein Rab2A (RAB2A), hemoglobin subunit beta (HBB), stomatin-like protein 2 (STOML2), alpha-crystallin B (CRYAB), S100A7, carbonic anhydrase 1 (CA1), TAR DNA-binding protein 43 (TARDBP), ruvB-like 2 (RUVBL2), and/or synaptotagmin binding cytoplasmic RNA interacting protein (SYNCRIP; also referred to as heterogeneous nuclear ribonucleoprotein Q).

In some embodiments, the polypeptide markers disclosed herein can have amino acid sequences as follows, or can have amino acid sequences that are at least 90% (e.g., at least 91%, 92%, 93%, 94%, 95%, 96%, 97%, 98%, or 99%) identical to the following exemplary sequences. A wild-type human MYL6B polypeptide can have the amino acid sequence set forth in NCBI Reference Sequence NP 002466 (e.g., version NP_002466.1). A wild-type human ENO3 polypeptide can have the amino acid sequence set forth in NCBI Reference Sequence NP_001967 (e.g., version NP_001967.3). A wild-type human MYH2 polypeptide can have the amino acid sequence set forth in NCBI Reference Sequence NP_060004 (e.g., version NP_060004.3). A wild-type human MYLPF polypeptide can have the amino acid sequence set forth in NCBI Reference Sequence NP_037424 (e.g., version NP_037424.2). A wild-type human MYH6 polypeptide can have the amino acid sequence set forth in NCBI Reference Sequence NP_002462 (e.g., version NP_002462.2). A wild-type human TPM2 polypeptide can have the amino acid sequence set forth in NCBI Reference Sequence NP_998839 (e.g., version NP_998839.1). A wild-type human FLNC polypeptide can have the amino acid sequence set forth in NCBI Reference Sequence NP_001449 (e.g., version NP_001449.3). A wild-type human RAB2A polypeptide can have the amino acid sequence set forth in NCBI Reference Sequence NP_002856 (e.g., version NP_002856.1). A wild-type human HBB polypeptide can have the amino acid sequence set forth in NCBI Reference Sequence NP_000509 (e.g., version NP_000509.1). A wild-type human STOML2 polypeptide can have the amino acid sequence set forth in NCBI Reference Sequence NP_038470 (e.g., version NP_038470.1). A wild-type human CRYAB polypeptide can have the amino acid sequence set forth in NCBI Reference Sequence NP_001276737 (e.g., version NP_001276737.1). A wild-type human S100A7 polypeptide can have the amino acid sequence set forth in NCBI Reference Sequence NP_002954 (e.g., version NP_002954.2). A wild-type human CA1 polypeptide can have the amino acid sequence set forth in NCBI Reference Sequence NP_001729 (e.g., version NP_001729.1). A wild-type human TARDBP polypeptide can have the amino acid sequence set forth in NCBI Reference Sequence NP_031401 (e.g., version NP_031401.1). A wild-type human RUVBL2 polypeptide can have the amino acid sequence set forth in NCBI Reference Sequence NP_006657 (e.g., version NP_006657.1). A wild-type human SYNCRIP polypeptide can have the amino acid sequence set forth in NCBI Reference Sequence NP_006363 (e.g., version NP_006363.4).

In some embodiments, expression of a polypeptide marker can be evaluated based on the level of the mRNA encoding the polypeptide. mRNAs for the polypeptide markers disclosed herein can have nucleotide acid sequences as follows, or can have nucleotide sequences that are at least 90% (e.g., at least 91%, 92%, 93%, 94%, 95%, 96%, 97%, 98%, or 99%) identical to the following exemplary sequences (noting that the thymine bases in the following Reference Sequences will be uracil in the mRNA sequences). A wild-type human MYL6B mRNA can have the nucleotide sequence set forth in NCBI Reference Sequence NM 002475 (e.g., version NM 002475.5). A wild-type human ENO3 mRNA can have the nucleotide sequence set forth in NCBI Reference Sequence NM_001976 (e.g., version NM_001976.5). A wild-type human MYH2 mRNA can have the nucleotide sequence set forth in NCBI Reference Sequence NM_017534 (e.g., version NM_017534.6). A wild-type human MYLPF mRNA can have the nucleotide sequence set forth in NCBI Reference Sequence NM_013292 (e.g., version NM_013292.5). A wild-type human MYH6 mRNA can have the nucleotide sequence set forth in NCBI Reference Sequence NM_002471 (e.g., version NM_002471.4). A wild-type human TPM2 mRNA can have the nucleotide sequence set forth in NCBI Reference Sequence NM_213674 (e.g., version NM_213674.1). A wild-type human FLNC mRNA can have the nucleotide sequence set forth in NCBI Reference Sequence NM_001458 (e.g., version NM_001458.5). A wild-type human RAB2A mRNA can have the nucleotide sequence set forth in NCBI Reference Sequence NM_002865 (e.g., version NM_002865.3). A wild-type human HBB mRNA can have the nucleotide sequence set forth in NCBI Reference Sequence NM_000518 (e.g., version NM_000518.5). A wild-type human STOML2 mRNA can have the nucleotide sequence set forth in NCBI Reference Sequence NM_013442 (e.g., version NM_013442.3). A wild-type human CRYAB mRNA can have the nucleotide sequence set forth in NCBI Reference Sequence NM_001289808 (e.g., version NM_001289808.2). A wild-type human S100A7 mRNA can have the nucleotide sequence set forth in NCBI Reference Sequence NM_002963 (e.g., version NM_002963.4). A wild-type human CA1 mRNA can have the nucleotide sequence set forth in NCBI Reference Sequence NM_001738 (e.g., version NM_001738.5). A wild-type human TARDBP mRNA can have the nucleotide sequence set forth in NCBI Reference Sequence NM_007375 (e.g., version NM_007375.4). A wild-type human RUVBL2 mRNA can have the nucleotide sequence set forth in NCBI Reference Sequence NM_006666 (e.g., version NM_006666.3). A wild-type human SYNCRIP mRNA can have the nucleotide sequence set forth in NCBI Reference Sequence NM_006372 (e.g., version NM_006372.5).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq c:\seql.txt -j c:\seq2.txt -p blastn -o c:\output.txt -q −1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seql.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence or by an articulated length (e.g., 100 consecutive amino acid residues from a sequence set forth in an identified sequence), followed by multiplying the resulting value by 100. For example, the reference human MYL6B amino acid sequence set forth in NCBI Reference Sequence NP_002466 is 208 residues in length. An amino acid sequence that has 200 matches when aligned with the reference sequence is 96.2 percent identical to the reference sequence (i.e., 200/208×100=96.2). It is noted that the percent sequence identity value is rounded to the nearest tenth. For example, 75.11, 75.12, 75.13, and 75.14 are rounded down to 75.1, while 75.15, 75.16, 7.17, 75.18, and 7.19 are rounded up to 7.2. It also is noted that the length value will always be an integer.

Lesional cells can be sampled via any appropriate method (e.g., biopsy, brushing, swabbing, scraping, or saliva collection). Any appropriate method can be used to determine if a mammal (e.g., a human) has elevated levels of one or more of the markers (e.g., polypeptide or mRNA) listed herein. The term “elevated level” as used herein with respect to the expression level of a polypeptide or mRNA marker refers to a level of expression of the marker by cells (e.g., oral squamous epithelial or stromal cells affected by LP) that is greater (e.g., at least 5, 10, 25, 35, 45, 50, 55, 65, 75, 80, 90, or 100 percent greater) than the median expression level of that marker in unaffected (e.g., adjacent “normal”) tissue or cells of the same type known not to have an elevated level of that marker from the same mammal, or in control oral LP mucosal cells from a mammal with oral LP known to follow a benign course. Appropriate methods for identifying cells as having an elevated level of one or more polypeptide markers as described herein include, without limitation, polypeptide-based assays such as antibody staining techniques or array hybridization assays using antibodies. Appropriate methods for identifying cells as having an elevated level of one or more mRNA markers as described herein include, without limitation, Northern blotting and quantitative reverse transcriptase-coupled polymerase chain reaction (RT-PCR).

Once a mammal (e.g., a human) is identified as having cells elevated levels of one or more of the markers as described herein, the mammal can be classified as having an increased risk of developing a malignancy such as SCC. For example, a human identified as having cells (e.g., oral squamous epithelial or stromal cells affected by LP) with increased levels of one or more (e.g., two, three, four, five, six, seven, eight, nine, ten, eleven, or all twelve) polypeptides selected from MYL6B, ENO3, MYH2, MYLPF, MYH6, TPM2, FLNC, RAB2A, HBB, STOML2, CRYAB, S100A7, CA1, TARDBP, RUVBL2, and SYNCRIP can be classified as being at increased risk of developing an oral cancer such as SCC. In some cases, a mammal (e.g., a human) identified as having cells (e.g., oral squamous epithelial or stromal cells affected by LP) that do not exhibit increased levels of one or more (e.g., two, three, four, five, six, seven, eight, nine, ten, eleven, or all twelve) polypeptides selected from MYL6B, ENO3, MYH2, MYLPF, MYH6, TPM2, FLNC, RAB2A, HBB, STOML2, CRYAB, S100A7, CA1, TARDBP, RUVBL2, and SYNCRIP can be classified as not being at increased risk of developing an oral cancer such as SCC.

Any combination of markers can be evaluated. For example, when increased levels of two polypeptide biomarkers are used to identify a mammal having oral LP and being at increased risk of malignancy, for example, the two polypeptides can be MYL6B and ENO3, MYL6B and MYH2, MYL6B and MYLPF, MYL6B and MYH6, MYL6B and TPM2, MYL6B and FLNC, MYL6B and RAB2A, MYL6B and HBB, MYL6B and STOML2, MYL6B and CRYAB, MYL6B and S100A7, MYL6B and CA1, MYL6B and TARDBP, MYL6B and RUVBL2, MYL6B and SYNCRIP, ENO3 and MYH2, ENO3 and MYLPF, ENO3 and MYH6, ENO3 and TPM2, ENO3 and FLNC, ENO3 and RAB2A, ENO3 and HBB, ENO3 and STOML2, ENO3 and CRYAB, ENO3 and S100A7, ENO3 and CA1, ENO3 and TARDBP, ENO3 and RUVBL2, ENO3 and SYNCRIP, MYH2 and MYLPF, MYH2 and MYH6, MYH2, and TPM2, MYH2 and FLNC, MYH2 and RAB2A, MYH2 and HBB, MYH2 and STOML2, MYH2 and CRYAB, MYH2 and S100A7, MYH2 and CA1, MYH2 and TARDBP, MYH2 and RUVBL2, MYH2 and SYNCRIP, MYLPF and MYH6, MYLPF and TPM2, MYLPF and FLNC, MYLPF and RAB2A, MYLPF and HBB, MYLPF and STOML2, MYLPF and CRYAB, MYLPF and S100A7, MYLPF and CA1, MYLPF and TARDBP, MYLPF and RUVLB2, MYLPF and SYNCRIP, MYH6 and TPM2, MYH6 and FLNC, MYH6 and RAB2A, MYH6 and HBB, MYH6 and STOML2, MYH6 and CRYAB, MYH6 and S100A7, MYH6 and CA1, MYH6 and TARDBP, MYH6 and RUVBL2, MYH6 and SYNCRIP, TPM2 and FLNC, TPM2 and RAB2A, TPM2 and HBB, TPM2 and STOML2, TPM2 and CRYAB, TPM2 and S100A7, TPM2 and CA1, TPM2 and TARDBP, TPM2 and RUVBL2, TPM2 and SYNCRIP, FLNC and RAB2A, FLNC and HBB, FLNC and STOML2, FLNC and CRYAB, FLNC and S100A7, FLNC and CA1, FLNC and TARDBP, FLNC and RUVBL2, FLNC and SYNCRIP, RAB2A and HBB, RAB2A and STOML2, RAB2A and CRYAB, RAB2A and S100A7, RAB2A and CA1, RAB2A and TARDBP, RAB2A and RUVBL2, RAB2A and SYNCRIP, HBB and STOML2, HBB and CRYAB, HBB and S100A7, HBB and CA1, HBB and TARDBP, HBB and RUVBL2, HBB and SYNCRIP, STOML2 and CRYAB, STOML2 and S100A7, STOML2 and CA1, STOML2 and TARDBP, STOML2 and RUVBL2, STOML2 and SYNCRIP, CRYAB and S100A7, CRYAB and CA1, CRYAB and TARDBP, CRYAB and RUVBL2, CRYAB and SYNCRIP, S100A7 and CA1, S100A7 and TARDBP, S100A7 and RUVBL2, S100A7 and SYNCRIP, CA1 and TARDBP, CA1 and RUVBL2, CA1 and SYNCRIP, TARDBP and RUVBL2, TARDBP and SYNCRIP, or RUVBL2 and SYNCRIP. Similarly, any combination of three, four, five, six, seven, eight, nine, ten, eleven, or all twelve of the aforementioned markers can be evaluated to determine whether a mammal with oral LP has an increased likelihood of developing malignancy.

As described herein, this document also provides methods and materials for treating a mammal identified as being at increased risk for developing malignancy. Any appropriate mammal identified as having LP and being at increased risk of malignancy can be administered one or more a topical or systemic immunosuppressive or immunomodulatory agents (e.g., a corticosteroid, dapsone, azathioprine, tacrolimus, mycophenolate mofetil, or a biologic agent). As described herein, mammals having oral LP who are likely to progress to malignancy (e.g., SCC) can be administered tailored therapies to achieve disease control while minimizing immunosuppression. Having the ability to use more aggressive systemic agents (e.g., azathioprine, tacrolimus, mycophenolate mofetil, or biologic agents) to achieve better disease control can allow clinicians and patients to proceed with treatment options that mitigate the risk for malignant transformation. In addition, mammals with increased risk for malignancy can undergo more regular surveillance (e.g., examination and/or biopsy of involved tissue on a more frequent basis) to detect changes of possible carcinoma development early in its course.

In some cases, one or more immunosuppressive or immunomodulatory agents (e.g., a corticosteroid, dapsone, azathioprine, tacrolimus, mycophenolate mofetil, or a biologic agent) can be administered to a mammal once or multiple times over a period of time ranging from days to months. In some cases, one or more immunosuppressive or immunomodulatory agents (e.g., a corticosteroid, dapsone, azathioprine, tacrolimus, mycophenolate mofetil, or a biologic agent) can be formulated into a pharmaceutically acceptable composition for administration to a mammal having oral LP that has been identified as having an elevated level of one or more polypeptide markers as described herein. For example, a therapeutically effective amount of an immunosuppressive or immunomodulatory agent (e.g., a corticosteroid, dapsone, azathioprine, tacrolimus, mycophenolate mofetil, or a biologic agent) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing one or more immunosuppressive or immunomodulatory agents (e.g., a corticosteroid, dapsone, azathioprine, tacrolimus, mycophenolate mofetil, or a biologic agent) can be designed for oral, topical, or parenteral (including subcutaneous, intramuscular, intravenous, and intradermal) administration. When being administered orally, a pharmaceutical composition can be in the form of a pill, tablet, or capsule. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

In some cases, a pharmaceutically acceptable composition containing one or more immunosuppressive or immunomodulatory agents (e.g., a corticosteroid, dapsone, azathioprine, tacrolimus, mycophenolate mofetil, or a biologic agent) can be administered locally or systemically. For example, a composition provided herein can be administered locally by intravenous injection or blood infusion. In some cases, a composition provided herein can be administered systemically, orally, or by injection to a mammal (e.g., a human).

Effective doses can vary depending on the severity of the oral LP, the route of administration, the age and general health condition of the subject, excipient usage, the possibility of co-usage with other therapeutic treatments, and the judgment of the treating physician.

An effective amount of a composition containing an immunosuppressive or immunomodulatory agent (e.g., a corticosteroid, dapsone, azathioprine, tacrolimus, mycophenolate mofetil, or a biologic agent) can be any amount that reduces the likelihood that the oral LP will progress to a malignancy without producing significant toxicity to the mammal. For example, an effective amount of an immunosuppressive or immunomodulatory agent (e.g., a corticosteroid, dapsone, azathioprine, tacrolimus, mycophenolate mofetil, or a biologic agent) can be from about 0.25 mg/kg to about 100 mg/kg (e.g., from about 0.3 mg/kg to about 11 mg/kg, from about 1 mg/kg to about 10 mg/kg, from about 2 mg/kg to about 10 mg/kg, from about 5 mg/kg to about 10 mg/kg, from about 6 mg/kg to about 10 mg/kg, from about 6 mg/kg to about 8 mg/kg, or from about 7 mg/kg to about 9 mg/kg). In some cases, from about 100 mg to about 1000 mg (e.g., from about 250 mg to about 1000 mg, from about 300 mg to about 1000 mg, from about 400 mg to about 1000 mg, from about 100 mg to about 900 mg, from about 100 mg to about 800 mg, from about 400 mg to about 800 mg, or from about 500 mg to about 700 mg) of an immunosuppressive or immunomodulatory agent (e.g., a corticosteroid, dapsone, azathioprine, tacrolimus, mycophenolate mofetil, or a biologic agent) can be administered to an average sized human (e.g., about 75-85 kg human) per administration (e.g., per daily or weekly administration) for about two to about twelve weeks or more.

If a particular mammal fails to respond to a particular amount, then the amount of the administered immunosuppressive or immunomodulatory agent(s) can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of an immunosuppressive or immunomodulatory agent (e.g., a corticosteroid, dapsone, azathioprine, tacrolimus, mycophenolate mofetil, or a biologic agent) can be any frequency that reduces the likelihood that the oral LP will progress to a malignancy without producing significant toxicity to the mammal. For example, the frequency of administration can be from about once a day to about once a month (e.g., from about once a week to about once every other week). The frequency of administration can remain constant or can be variable during the duration of treatment. A course of treatment with a composition containing one or more immunosuppressive or immunomodulatory agents (e.g., a corticosteroid, dapsone, azathioprine, tacrolimus, mycophenolate mofetil, or a biologic agent) can include rest periods. For example, a composition containing an immunosuppressive or immunomodulatory agent (e.g., a corticosteroid, dapsone, azathioprine, tacrolimus, mycophenolate mofetil, or a biologic agent) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., oral LP) may require an increase or decrease in administration frequency.

An effective duration for administering a composition containing one or more immunosuppressive or immunomodulatory agents (e.g., a corticosteroid, dapsone, azathioprine, tacrolimus, mycophenolate mofetil, or a biologic agent) can be any duration that reduces the likelihood that the oral LP will progress to a malignancy without producing significant toxicity to the mammal. In some cases, the effective duration can vary from several days to several months. In general, the effective duration for reducing the likelihood that oral LP will progress to a malignancy can range from about six weeks to about six months. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to the condition being treated (e.g., oral LP) can be monitored. Any appropriate method can be used to determine whether or not a mammal's likelihood of developing a malignancy is being delayed or reduced. For example, cells can be assessed following administration of an immunosuppressive or immunomodulatory agent (e.g., a corticosteroid, dapsone, azathioprine, tacrolimus, mycophenolate mofetil, or a biologic agent) to determine if the treatment reduced the level of one or more of the polypeptide markers in the cells, as compared to previously measured levels.

The invention will be further described in the following example, which does not limit the scope of the invention described in the claims.

### EXAMPLE

Six oral mucosal biopsies of oral LP with eventual malignant transformation and six cases of oral LP with a benign course (at least 5 years of follow-up with no malignancy) were studied. Cases showing dysplasia were excluded. Laser capture microdissection-assisted liquid-chromatography-based tandem mass spectrometry was performed on archived, formalin-fixed-paraffin embedded tissue. Protein expression in each group was compared by total spectral counts and ion peak intensities. Differentially-expressed proteins were analyzed by the STRING DB platform, and protein biology function was investigated by literature search.

Proteins that were differentially expressed (>3-fold expression; p<0.05; false discovery rate<1%) by total spectral counts and ion peak intensities were recorded. Sixty-three proteins were identified as being overexpressed in the group of oral LP with malignant transformation. Upregulation of the p53 pathway, a pathway involved in carcinogenesis, was suggested by overexpression of MYL6B, a mediator of p53 degradation, and ENO3, the gene for which is located next to P53. Candidate biomarkers of interest also included cytoskeletal proteins (MYH2, MYL6B, TPM2 and FLNC). Increased expression of STOML2, CRYAB, and S100A7, reported elsewhere as potential biomarkers for oral carcinomas, also was observed, as was increased expression of several novel proteins that may be biomarkers, including CA1, TARDBP, RUVBL2, and SYNCRIP.

Deregulation of cytoskeletal signaling was suggested by differential expression of certain myosins (MYL6B, MYH2 and MYH6) and TPM2. As there is an increased awareness of the role of myosins and tropomyosins in tumorigenesis, this finding is of particular interest. As examples, the degradation of p53 in hepatocellular carcinoma seems to be dependent on MYL6B and amplification of TPM2 has been found in oral squamous cell carcinoma. In addition, STOML2 and S100A7 have been shown to be increased in dysplastic oral lesions that progressed to squamous cell carcinoma, and are considered key players in oral carcinogenesis. As cases displaying dysplasia were excluded from the present studies, the increased expression of STOML2 and S100A7 indicated that there are molecular alterations even before histopathologic changes are apparent, and that oral SCC associated with LP may share carcinogenesis pathways with conventional SCC. Further, increased expression of RAB2A, a protein activated through S100A7, also was observed in the present studies.

Thus, the studies described herein revealed proteins that appear to be of prognostic value when assessed in tissue showing LP (e.g., oral LP).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a mammal having lichen planus (LP) as being at increased risk of developing cancer, said method comprising identifying a mammal with LP as having cells with increased expression of one or more markers, wherein said one or more markers comprise one or more of myosin light chain 6B (MYL6B), beta-enolase 3 (ENO3), myosin-2 (MYH2), myosin regulatory light chain 2 (MYLPF), myosin-6 (MYH6), tropomyosin 2 (TPM2), filamin-C (FLNC), Ras-related protein Ras2A (RAS2A), hemoglobin subunit beta (HBB), stomatin-like protein 2 (STOML2), alpha-crystallin B (CRYAB), S100A7, carbonic anhydrase 1 (CA1), TAR DNA-binding protein 43 (TARDBP), ruvB-like 2 (RUVBL2), and synaptotagmin binding cytoplasmic RNA interacting protein (SYNCRIP), thereby identifying said mammal as being at increased risk of developing cancer.

2. The method of claim 1, wherein said mammal is a human.

3. The method of claim 1, wherein said LP is oral LP.

4. The method of claim 1, wherein said cells are within a cytology brushing sample.

5. The method of claim 1, wherein said cells are squamous epithelial or stromal cells affected by said LP.

6. The method of claim 1, wherein said identifying comprises measuring the level of protein for said one or more markers.

7. The method of claim 1, comprising identifying the mammal with oral LP as having cells with increased expression of two or more of said markers.

8. A method for treating a mammal having LP, said method comprising:
(a) identifying said mammal as having cells with increased expression of one or more markers, wherein said one or more markers comprise one or more of MYL6B, ENO3, MYH2, MYLPF, MYH6, TPM2, FLNC, RAB2A, HBB, STOML2, CRYAB, S100A7, CA1, TARDBP, RUVBL2, and SYNCRIP, and
(b) administering to said mammal an immunosuppressive or immunomodulatory agent.

9. The method of claim 8, wherein the mammal is a human.

10. The method of claim 8, wherein the LP is oral LP.

11. The method of claim 8, wherein said cells are within a cytology brushing sample.

12. The method of claim 8, wherein said cells are squamous epithelial or stromal cells affected by said LP.

13. The method of claim 8, wherein said identifying comprises measuring the level of protein for said one or more markers.

14. The method of claim 8, comprising identifying the mammal with LP as having cells with increased expression of two or more of said polypeptides.

15. The method of claim 8, wherein said immunosuppressive or immunomodulatory agent comprises a corticosteroid, dapsone, azathioprine, tacrolimus, mycophenolate mofetil, or a biologic agent.

16. A method for treating a mammal having LP, said method comprising administering, to a mammal identified as having cells with increased expression of one or more markers selected from the group consisting of MYL6B, ENO3, MYH2, MYLPF, MYH6, TPM2, FLNC, RAB2A, HBB, STOML2, CRYAB, S100A7, CA1, TARDBP, RUVBL2, and SYNCRIP, an immunosuppressive or immunomodulatory agent.

17. The method of claim 16, wherein said mammal is a human.

18. The method of claim 16, wherein said LP is oral LP.

19. The method of claim 16, wherein the cells are squamous epithelial or stromal cells affected by said LP.

20. The method of claim 16, wherein said mammal with LP was identified as having cells with increased expression of two or more of said markers.

21. The method of claim 16, wherein said immunosuppressive or immunomodulatory agent comprises a corticosteroid, dapsone, azathioprine, tacrolimus, mycophenolate mofetil, or a biologic agent.
